(19) United States
(12) Patent Application Publication
(10) Pub. No.: US 2006/0210422 A1
(43) Pub. Date: Sep. 21, 2006

(54) Apparatus and method for sterile interconnection of lines transferring sterile fluids

(76) Inventor: Roderick R. Roberts, Redding, CA (US)

Correspondence Address: R. MICHAEL WEST LAW OFFICES OF;R. MICHAEL WEST, A PROFESSIONAL CORPORATION, 455 CAPITOL MALL; SUITE 405, SACRAMENTO, CA, 95814-4603, US

(21) Appl. No.: 11/082,382

(22) Filed: Mar. 16, 2005

(51) Int. Cl. A61L 2/20

(52) U.S. Cl. 422/28; 422/292

(57) ABSTRACT

An apparatus and method to effect a sterile interconnection of lines for transferring sterile medical fluids from a supply container to a fluid utilization device or a fluid storage container. A flexible, transparent, sterile connection bag is provided with at least three inlet/outlet ports. Previously sterilized free ends of the incoming and outgoing fluid lines, provided with connectors, are inserted into the bag through two of the ports and sealed therewith. A gas sterilization line is inserted into the bag through the third port. A sterilizing gas, such as ozone, is introduced into the bag to re-sterilize the lines and the connectors. Through visual inspection and external hand manipulation of the bag and its contents, the two lines are joined within the bag by the line connectors. Sterile fluid transfer through the lines is then undertaken.

### BACKGROUND OF THE INVENTION

1. Field of the Invention

The invention relates generally to devices and methods for transferring sterile fluids from a fluid supply container to a fluid utilization device or a fluid storage container while maintaining the sterility of that fluid. More particularly, the invention pertains to an apparatus and a method in which: at least two sterile fluid transfer lines are inserted into a flexible, sterile connection bag which is either transparent or includes a transparent viewing window; a sterilizing gas, such as ozone, is introduced into the bag; the fluid transfer lines are manually interconnected within the bag by external hand manipulation; and, a sterile fluid is passed from one fluid transfer line to the other fluid transfer line.

2. Description of the Prior Art

Transferring sterile fluids from one vessel or container to another is a daily occurrence in hospitals, pharmacies, veterinary clinics, blood banks, research facilities, and medical supply companies. For example, bio-technology and bio-pharmaceutical companies utilize large volumes of sterile fluids when conducting research and producing medicine. In the course of this research and production, sterile fluids must be transferred from a large storage container to another container directly associated with the sterile fluid utilization. If contaminating bacteria is introduced into the means for fluid transfer, an entire research project or a batch of medicine in production may be lost.

The containers which store and which receive transferred sterile fluids vary from plastic bags to stainless steel vessels, also known as bioreactors. Sterilized plastic bags are typically disposed of after one use. Stainless steel vessels, on t he other hand, are made the subject of multiple uses over a period of time. Therefore, vessels are sterilized, usually by steam, prior to use for storing or receiving sterile fluids. Tubes or hoses attached to these vessels by means of specialized connectors, are also sterilized at the same time. Fluid transfer tubes or hoses have protective covers over their ends to maintain sterility until their ultimate attachment to another vessel or a bag. Currently, the entire system must be sterilized, or re-sterilized, after all of the vessel, bag, and line connections have been made. Owing to the size of these components and the overall system, re-sterilization is a time-consuming and exacting practice. If re-sterilization could be limited only to interconnections made between fluid line tubes or hoses, much time and effort could be saved.

In U.S. Pat. No. 4,242,310, issued to Greffet al., an apparatus designed to effect a sterile interconnection of the ends of sterile fluid lines is shown. In this arrangement, a rigid transparent housing having a hinged cover is provided. A flexible membrane within one wall of the cover allows sterile entry of a user's hand into the interior of the housing. Multiple fluid supply lines enter the housing, from fluid bags or sacks supported in an adjacent rack. An outflow fluid line also enters the housing, for interconnection to one of the two fluid supply lines. A sterilizing chemical agent or steam is introduced into the housing for sterilizing the various lines.

However, the need still exists for an inexpensive yet fault-proof means for maintaining sterility throughout a sterile fluid transfer system, where that system includes connections or junctures between fluid transfer lines

There is also a need for a lightweight, disposable, and inexpensive sterile connection bag or container where sterile interconnections can be made between incoming fluid lines and outgoing fluid lines.

There is also a need for a sterile connection bag or container where sterile interconnections can be made between fluid lines through a combination of visual inspection and direct manual manipulation of fluid lines and their connectors through the wall of the sterile container.

These and other objects of the present invention will be described in the drawings and in the detailed description of the preferred embodiment set forth below.

### SUMMARY OF THE INVENTION

The apparatus of the present invention comprises a flexible sterile connection bag or container, having a sidewall with a transparent portion therein. Also provided in the connection bag sidewall are a gas sterilization port, an incoming port, and an outgoing port . The free end of an incoming fluid line is passed through the incoming port, so that it extends into the interior volume of the connection bag. The free end of an outgoing fluid line is passed through the outgoing port, so that it extends into the interior volume of the connection bag. One of the free ends of the fluid lines may be provided with a line connector prior to its insertion into the connection bag. Alternatively, the line connector may independently be inserted into the connection bag through one of the open ports.

A gas sterilization generator has an outlet connected to one end of a gas sterilization line. The other free end of the gas sterilization line is passed through the gas sterilization port to extend into the interior volume of the connection bag. The sterilizing gas is effective to re-sterilize all line and connector components within the interior volume of the connection bag.

Following re-sterilization, the user of the apparatus views the line and connector components through the transparent portion of the sidewall. Utilizing hand manipulation applied through the flexible bag, the user then removes the protective caps on the free ends of the fluid lines and the connector. Similarly, the incoming and outgoing fluid lines are manually connected, and the fluid lines are ready for sterile transfer of the fluid.

### BRIEF DESCRIPTION OF THE DRAWINGS

FIG. 1 is a perspective view of a fluid supply container, an ozone generator, a fluid storage bag, interconnected to the sterile connection bag of the present invention;

FIG. 2 is a perspective view of the sterile connection bag, showing the incoming fluid line, and outgoing fluid line, and the ozone line

FIG. 3 is a view as in FIG. 2, but showing the ozone line connected to an inlet line, and the fluid lines inserted into the sterile connection bag with their protective caps removed;

FIG. 4 is a cross-sectional view, taken on the line 4-4 in FIG. 5;

FIG. 5 is a detailed view of a friction-fitted port plug in proximity to a fluid line;

FIG. 6 is detailed view of an alternative construction for a port plug including external threads, the plug being shown installed over a fluid line;

FIG. 7 is a view as in FIG. 3, but showing the fluid lines interconnected with a standard male and female line coupler;

FIG. 8 is an alternative construction for the sterile connection bag, showing another port accommodating a second incoming fluid line and using barbed line couplers; and,

FIG. 9 is a view as in FIG. 8, but showing the outgoing fluid line now interconnected to the second incoming fluid line.

### DETAILED DESCRIPTION OF THE PREFERRED EMBODIMENT

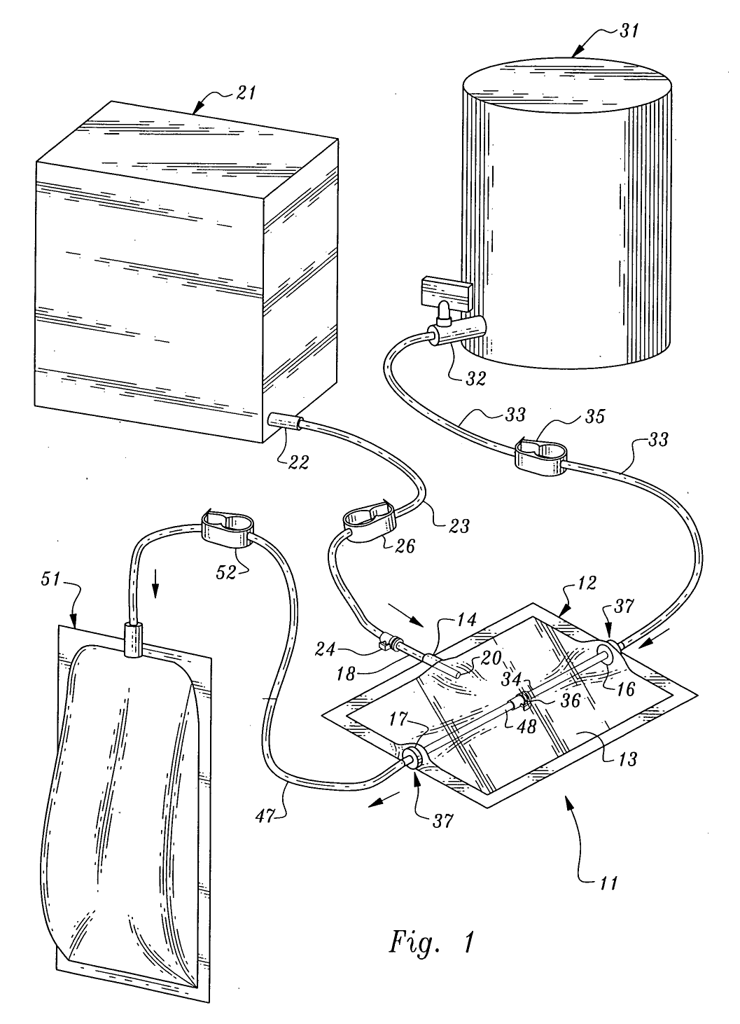

Turning now to the drawings, and in particular to FIG. 1, the apparatus 11 of the present invention includes a flexible sterile connection bag 12. Connection bag 12 is preferably made entirely from a transparent material, such as a plastic, vinyl, or polyvinyl. However, to utilize the apparatus 11 effectively, it is only necessary that a sidewall of bag 12 have at least a portion 13 which is transparent, so as to enable the user to view the line and connector components within the contained volume of bag 12.

The connection bag 12 includes three ports which penetrate through its sidewall and extend into the inner, contained volume of the bag. These ports comprise a gas sterilization port 14, an incoming port 16, and an outgoing port 17. Once gas and fluid lines are installed in these ports, as explained more fully below, the connection bag 12 is hermetically sealed.

A tube stub 18 is passed through gas sterilization port 14, and a snug fit ensures a reasonably tight seal therewith. Stub 18 includes a male connector 19 on its exterior end, while its free end 20 extends into the contained volume of the connection bag 12. A gas sterilization generator 21 has a gas outlet 22 connected to one end of a gas sterilization line 23. Gas sterilization generator is a commercially available apparatus, preferably producing ozone or other gas capable of creating a sterilizing environment in a sealed container. A female connector 24 is provided on the other end of line 23, for ready interconnection to male connector 19. A conventional line clamp 26 may also be provided on line 23, to control the discharge of sterilizing gas through the line. Typically, protective caps 27 and 28 are respectively provided over connectors 19 and 24 to maintain the sterility of their connection portions up to the time the interconnection between them is made.

It may also be desirable, under certain conditions, to provide a gas sterilization line 29 which extends continuously from the outlet 22 of gas sterilization generator 21, directly into the contained volume of bag 12. In this arrangement, as shown in FIGS. 8 and 9, the line 29 is simply inserted through port 14, and no external male and female line connectors are used. Line 29 may also be permanently connected to and sealed within port 14, so that the bag 12 would effectively be pre-fitted with a gas sterilization line.

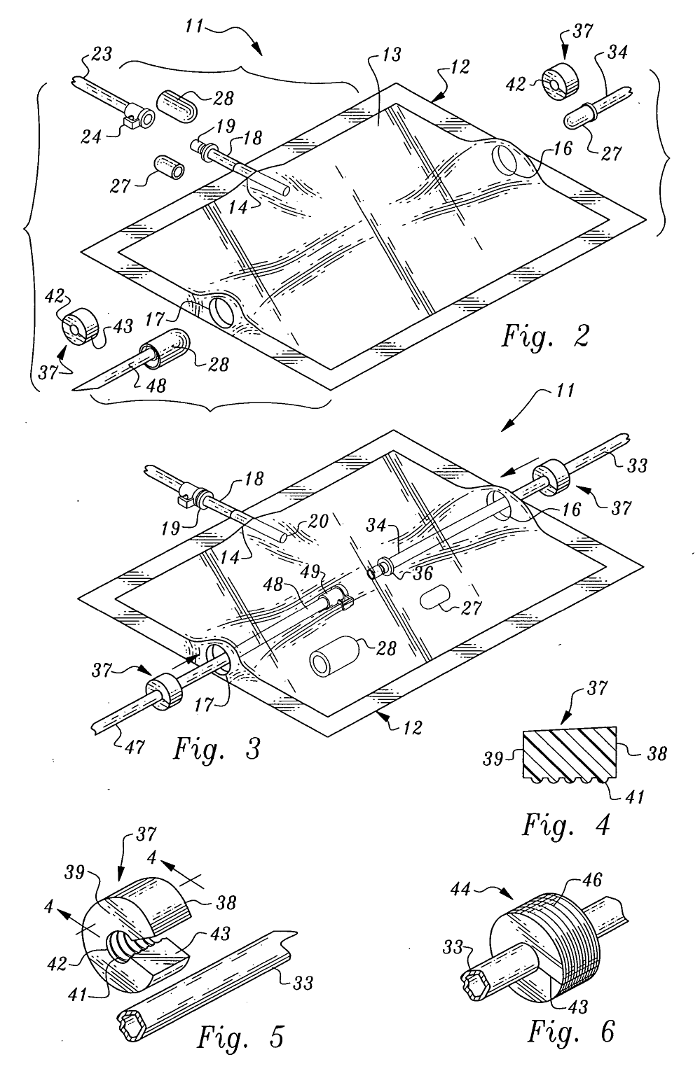

A sterilized fluid supply container 31 includes a discharge valve 32. Container 31 may be a stainless steel vessel, a tank, or a plastic bag, depending upon the sterilized fluid and the storage requirements. An incoming fluid line 33 has one end connected to discharge valve 32 and a free end 34 fitted with a male connector 36. Male connector 36 is pre-sterilized and fitted with a protective cap 27, as shown in FIG. 2. Protective cap 27 remains on connector 36 until the final connection step within connection bag 12 is undertaken. A line clamp 35 may be provided over line 33, further to control the flow of fluid therethrough.

A friction-fitted port plug 37 is provided to ensure a tight seal between fluid line 33 and incoming port 16. Plug 37 is preferably made from a resilient material which is capable of being compressed to some degree, as it is inserted into incoming port 16. To facilitate its installation, plug 37 is tapered slightly in its exterior diameter, from outer side 38 to inner side 39, as shown in FIG. 4. Plug 37 is provided with ribs 41 around a tube aperture 42 to form a tight seal over line 33. A slit 43 is provided in the side of plug 37, to ensure easy installation over line 33. As shown in FIG. 3, with the free end 34 of line 33 within the contained volume of bag 12, plug 37 is installed over line 33 and then pressed snugly into incoming port 16.

As an alternative construction, a port plug 44 including external threads 46 on its outer periphery may be employed as well. This construction also includes a slit 43 and ribs 41 (not shown) so it forms a tight seal and is readily installed over line 33. In addition, incoming port 16 must be provided with internal threads (not shown) so that plug 44 may be threadably engaged within port 16 while securing line 33 within bag 12.

It is apparent that many other constructions may be used to effect the required airtight interconnection between the fluid line and the port interconnection. For example, an internally threaded cap which passes the fluid line may be adapted to mate with an externally threaded port extension. Also, a snap-on male/female connector system provided with sealing 0-rings may be employed. Both of these connection arrangements are conventional and well known in the art, so no further description is needed.

On the other side of connection bag 12, an outgoing fluid line 47 is inserted through outgoing port 17, with its free end 48 extending into the contained volume of connection bag 12. A female connector 49, fitted with a protective cap 28, is provided on free end 48. The other end of outgoing fluid line 47 extends to a fluid storage bag 51. Depending upon the particular application, a fluid storage vessel or a fluid utilization device may be substituted for fluid storage bag 51. A line clamp 52 may be provided over line 47 to control outgoing fluid flow. As with the installation of line 33 within incoming port 16, a port plug 37 is fitted over outgoing fluid line 47 and installed within outgoing port 17 to ensure a tight seal therebetween.

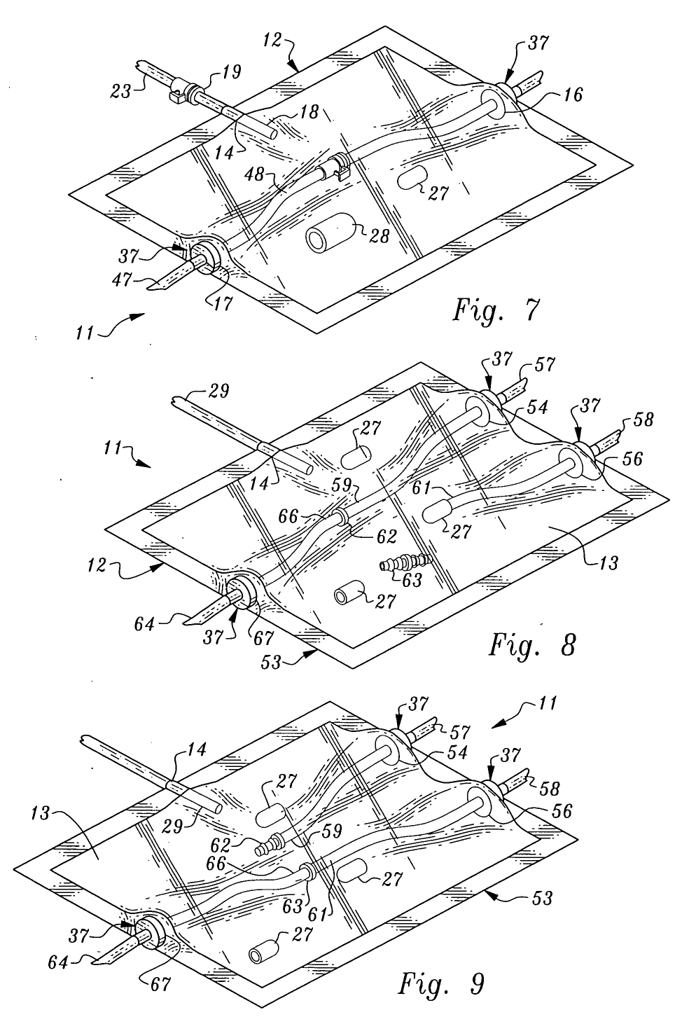

At this juncture, sterilizing gas produced by the generator 21 is introduced into the contained volume of the bag, providing in effect a re-sterilization and maintenance of sterilization, of all lines and components therein. This process may take some ten to fifteen minutes to complete. Once re-sterilization is complete, the actual interconnection between the incoming fluid line 33 and the outgoing fluid line 47 is made. In preparation, while viewing the components through the transparent portion of the bag 12, the user manually grips the protective caps 27 and 28 and removes them from their respective connectors. Owing to the flexibility and at least partial transparency of the bag 12, manually gripping and manipulating components within the contained volume of the bag is an easy process for the user. With the protective caps removed, male connector 36 and female connector 49 are interconnected. See, FIG. 7.

After the fluid line interconnection is complete, the required valves and clamps are opened to allow passage of the sterilized fluid from the container 31, through the incoming and outgoing lines 33 and 47, to the fluid storage bag 51.

Several alternative features are shown in the connection bag 53, illustrated in FIGS. 8 and 9. Connection bag 53 includes a gas sterilization port 14 and its associated gas sterilization line 29 leading to a gas sterilization generator 21 as previously described. However, connection bag 53 includes first incoming port 54 and second incoming port 56. A first incoming line 57 and a second incoming line 58 are also provided, along with their respective port plugs 37. Line 57 has a free end 59 and line 58 has a free end 61, both free ends extending into the contained volume of bag 53. A barbed connector 62 is provided on free end 59, and a barbed connector 63 is provided on free end 61. Preferably, barbed connectors 62 and 63 are pre-installed on lines 57 and 58, sterilized, and then fitted with protective caps 27 before the lines are inserted into bag 12.

An outgoing line 64 having a free end 66 is installed through outgoing port 67, in a manner identical to that previously described. Outgoing line is connected to a fluid tank, bag, or fluid utilization apparatus (not shown). Ozone or other sterilizing gas is introduced into the contained volume of bag 53 until all components and the lines are re-sterilized. As set forth above, owing to the transparent and flexible characteristics of the bag 53, the user is capable of viewing the internal components and lines, while grasping and manipulating the components and lines for selective interconnecttion. Then, protective caps 27 are removed from barb 62 and free end 66, and barb 62 is then inserted into free end 66 of outgoing line 64. See, FIG. 8. Finally, fluid is allowed to pass in a sterilized condition, from incoming line 57 to outgoing line 64.

If the supply of fluid provided to incoming line 57 is exhausted, or if a different fluid is desired to be provided to outgoing line 64, a simple line changeover is undertaken. Sterilizing gas may again be introduced into bag 53, to ensure re-sterilization of the contained lines and components. Again manipulating the contained lines and components, the user removes the protective cap 27 from barb 63 or from free end 61, withdraws free end 66 from barb 62, and inserts free end 66 into barb 63, as shown in FIG. 9. Then, fluid flow can begin, transferring fluid from incoming line 58 through outgoing line 64.

What is claimed is:

1. An apparatus for the sterile interconnection of lines transferring sterile fluids, comprising:
a. a flexible sterile connection bag having a gas sterilization port, an incoming port, and an outgoing port passing through a wall of said bag;
b. an incoming fluid line passing through said incoming port and having a free end extending into an interior volume of said bag;
c. an outgoing fluid line passing through said outgoing port and having a free end extending into an interior volume of said bag;
d. line connector means for coupling said end of said incoming fluid line and said end of said outgoing fluid line, within said interior volume of said bag;
e. a gas sterilization generator having an outlet discharging sterilizing gas;
f. a gas sterilization line having one end connected to said outlet of said gas sterilization generator, and the other end passing through said gas sterilization port and extending into said interior volume of said bag, said sterilizing gas being effective to maintain the sterility of said incoming fluid line, said outgoing fluid line, and said line connector means within said interior volume of said sterile connection bag.

2. An apparatus as in claim 1 in which at least a portion of said wall of said sterile connection bag is transparent.

3. An apparatus as in claim 1 in which said sterile connection bag is transparent.

4. An apparatus as in claim 1 in which said gas sterilization generator produces ozone.

5. An apparatus as in claim 1 in which said line connector means comprises a male connector and a female connector interconnecting said free end of said incoming fluid line and said free end of said outgoing fluid line.

6. An apparatus as in claim 1 in which said line connector means comprises a tube having opposing barbed ends sized and configured for snug installation into said free end of said incoming fluid line and said free end of said outgoing fluid line.

7. An apparatus as in claim 1 further including plugs within said incoming port and said outgoing port, said plugs having an aperture passing therethrough for accommodating said incoming line and said outgoing line.

8. An apparatus as in claim 7 in which said plugs have an outer periphery and a slit extending from said outer periphery to said aperture.

9. An apparatus as in claim 1 in which said free ends of said incoming fluid line and said outgoing fluid line are pre-sterilized and provided with removable caps, before said lines are passed through said incoming port and said outgoing port.

10. An apparatus as in claim 9 in which said caps are removed within said interior volume of said before said line connector means is used to couple said ends of said incoming and outgoing lines.

11. An apparatus for the sterile interconnection of lines transferring sterile fluids, comprising:
a. a flexible sterile connection bag having a gas sterilization port, an incoming port, and an outgoing port passing through a wall of said bag;
b. an incoming fluid line passing through said incoming port and having a free end extending into an interior volume of said bag;
c. an outgoing fluid line passing through said outgoing port and having a free end extending into an interior volume of said bag;
d. a coupler connecting said free end of said incoming fluid line and said free end of said outgoing fluid line, within said interior volume of said bag;
e. a gas sterilization generator having an outlet discharging sterilizing gas;
f. a gas sterilization line having one end connected to said outlet of said gas sterilization generator, and the other end passing through said gas sterilization port and extending into said interior volume of said bag, said sterilizing gas being effective to maintain the sterility of said incoming fluid line, said outgoing fluid line, and said line connector means within said interior volume of said sterile connection bag.

12. An apparatus as in claim 11 in which at least a portion of said wall of said sterile connection bag is transparent.

13. An apparatus as in claim 11 in which said sterile connection bag is transparent.

14. An apparatus as in claim 11 in which said gas sterilization generator produces ozone.

15. An apparatus as in claim 11 further including plugs within said incoming port and said outgoing port, said plugs having an aperture passing therethrough for accommodating said incoming line and said outgoing line.

16. An apparatus for the sterile interconnection of lines transferring sterile fluids, comprising:
a. a flexible sterile connection bag having a gas sterilization port, an incoming port, and an outgoing port passing through a wall of said bag;
b. an incoming fluid line passing through said incoming port and having a free end extending into an interior volume of said bag;
c. an outgoing fluid line passing through said outgoing port and having a free end extending into an interior volume of said bag;
d. a coupler connecting said free end of said incoming fluid line and said free end of said outgoing fluid line, within said interior volume of said bag;
e. means effective to maintain the sterility of said incoming fluid line, said outgoing fluid line, and said line connector means within said interior volume of said sterile connection bag.

17. An apparatus as in claim 16 in which said means effective to maintain sterility comprises a gas sterilization generator having an outlet discharging sterilizing gas, and a gas sterilization line having one end connected to said outlet of said gas sterilization generator, and the other end passing through said gas sterilization port and extending into said interior volume of said bag.

18. An apparatus as in claim 16 in which at least a portion of said wall of said sterile connection bag is transparent.

19. An apparatus as in claim 16 in which said sterile connection bag is transparent.

20. An apparatus as in claim 16 in which said gas sterilization generator produces ozone.

21. A method for the sterile interconnection of lines transferring sterile fluids, comprising:
a. providing a flexible sterile connection bag having a gas sterilization port, an incoming port, and an outgoing port passing through a wall of said bag;
b. passing a sterilized free end of an incoming fluid line through said incoming port into an interior volume of said bag;
c. passing a sterilized free end of an outgoing fluid line through said outgoing port into an interior volume of said bag;
d. introducing a sterilant gas into said interior volume of said sterile bag, re-sterilizing said incoming fluid line and said outgoing fluid line; and,
e. coupling said free ends of said incoming fluid line and said outgoing fluid line within said interior volume of said bag by hand manipulation of said bag and said lines.
